(19) United States
(12) Patent Application Publication
(10) Pub. No.: US 2021/0361808 A1
(43) Pub. Date: Nov. 25, 2021

(54) Disinfecting Enclosure System

(71) Applicant: Radhouane Ben Amor, Pickering (CA)

(72) Inventor: Radhouane Ben Amor, Pickering (CA)

(21) Appl. No.: 16/880,421

(22) Filed: May 21, 2020

(51) Int. Cl. A61L 2/24; A61L 2/00

(52) U.S. Cl. CPC A61L 2/24; A61L 2/0047; A61L 2/0088; A61L 2202/16; A61L 2202/122; A61L 2202/14; A61L 2202/15; A61L 2202/11

(57) ABSTRACT

A system for disinfecting; the system includes an enclosure, a first disinfecting assembly, a second disinfecting assembly and a power source. The first disinfecting assembly utilizes ultraviolet light and the second disinfecting assembly utilizes a disinfecting solution. The system is useful for disinfecting a body of an individual, their clothing, accessories, etc. upon entry to a building, or upon exit of the building.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of disinfecting systems of existing art and more specifically relates to an enclosure for disinfecting an individual.

### RELATED ART

The spread of bacteria, viruses and other pathogens is a known problem in areas of high foot traffic such as grocery stores, schools, offices, hospitals, etc. As such, keeping these buildings sanitized is of great importance. The importance of this is enhanced during times of epidemics and pandemics where viruses are easily spread from human to human—particularly when an individual can be an asymptomatic carrier of the virus, as was seen in the COVID-19 pandemic.

These buildings typically employ disinfection and sanitization methods such as hand sanitizer stations, the disinfection/sterilization of items between uses, the disposal of used items, etc. However, these methods only aim to combat the spread of pathogens once they are already in the building. They do not prevent the entrance of pathogens on an individual's body's, clothing, accessories, etc. Thus, a suitable solution is desired to provide a means of disinfecting an individual upon entrance or exit of a building.

### SUMMARY OF THE INVENTION

In view of the foregoing disadvantages inherent in the known disinfecting art, the present disclosure provides a novel disinfecting enclosure system. The general purpose of the present disclosure, which will be described subsequently in greater detail, is to provide a system for disinfecting an individual upon entrance to a building, and/or upon exit of a building, to prevent the spread of pathogens carried on their person.

A system for disinfecting is disclosed herein. The system for disinfecting includes an enclosure which may be defined by at least a roof, a first side wall, and a second side wall opposite the first side wall, an entrance and an exit. The enclosure may be sized to accommodate a human-user.

A first disinfecting assembly may be attached about an inner-surface of the enclosure. The first disinfecting assembly may include a light source emitting shortwave ultraviolet light configured to disinfect at least a portion of the human-user. Further, a second disinfecting assembly may be located about the inner-surface of the enclosure. The second disinfecting assembly may include a receptacle housing a disinfecting solution, and a dispensing means in communication with the receptacle. The dispensing means may be configured to selectively dispense a predetermined amount of disinfecting solution over the human-user (or portion of). In addition, a power source may be provided and configured to provide power to the system.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a disinfecting enclosure system, constructed and operative according to the teachings of the present disclosure.

FIG. 1 is a side front view of the system for disinfecting during an ‘in-use’ condition, according to an embodiment of the disclosure.

FIG. 2A is a top view of the system for disinfecting of FIG. 1, according to an embodiment of the present disclosure.

FIG. 2B is a side view of the system for disinfecting of FIG. 1, according to an embodiment of the present disclosure.

FIG. 3 is a side front view of the system for disinfecting of FIG. 1, according to an embodiment of the present disclosure.

FIG. 4 is a front top view of the system for disinfecting of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a disinfecting system and more particularly to a disinfecting enclosure system used to disinfect/sanitize a body (or at least a portion of the body), clothing, accessories of an individual, or other item upon entrance to a building, or upon exit of a building. This is designed to prevent bacteria, viruses, or other pathogens being carried on the individual from entering the building, and thus, reduce the spread of the bacteria, viruses, or other pathogens. The disinfecting enclosure system may be placed at any point of access to the building such as a hospital, grocery store, schools, offices, construction sites, residential or commercial buildings.

Generally, disclosed is a disinfecting station including an enclosure sized to accommodate a human-user; and an entrance and an exit for entrance and exit to the enclosure. The enclosure may include aluminum cross braces and aluminum tube framing, and a roof made from flame resistant board. Located within the enclosure may be an ultraviolet light disinfecting unit including a light source emitting the ultraviolet light that is housed within a vandal proof housing to protect the light source. Further, a disinfecting spray unit may be located within the housing and include spray nozzles and a 15-gallon container for holding disinfecting mixture. The ultraviolet light disinfecting unit and the disinfecting spray unit disinfect the human-user in the enclosure. The spray nozzles may be located on at least one side of the enclosure and the roof of the enclosure. The spray nozzles may distribute the disinfecting mixture as a mist or fog.

A motion sensor may be attached about the entrance of the enclosure and in communication with the ultraviolet light disinfecting unit and the disinfecting spray unit. When the human-user enters the enclosure, the motion sensor will detect the human-user; the ultraviolet light disinfecting unit and the disinfecting mixture spray unit may then actuate to disinfect the human-user. To provide power to the station, there may be a battery unit including batteries and a housing assembly located outside of the enclosure. The enclosure may further include casters to provide portability to the enclosure. The casters may each include locking brakes to provide stability when the human-user is within the enclosure.

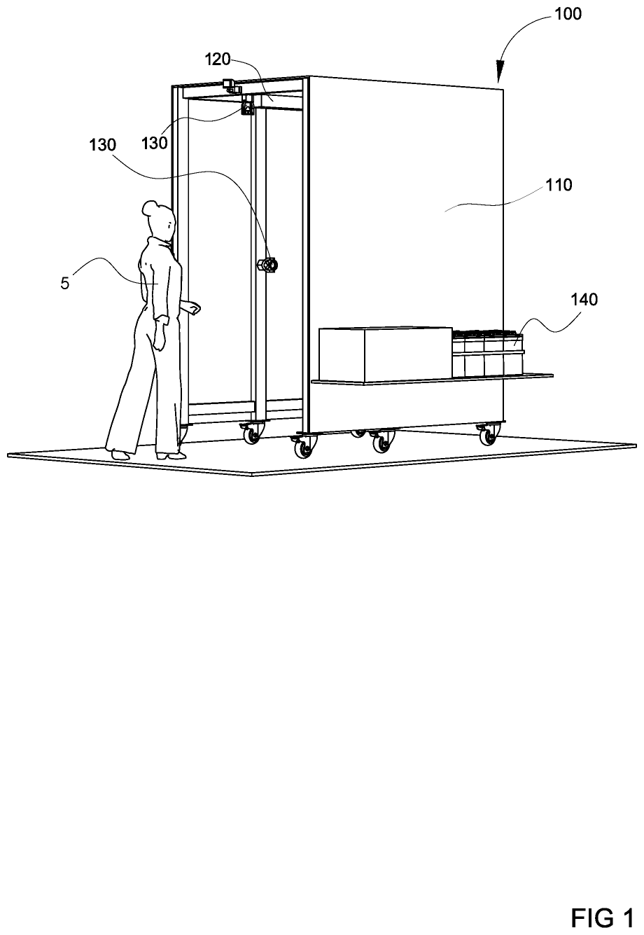

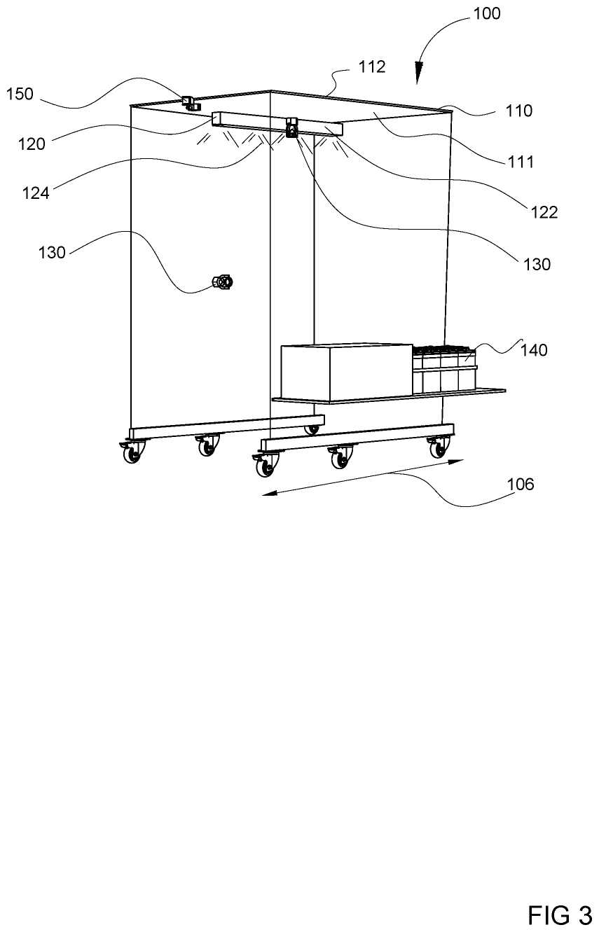

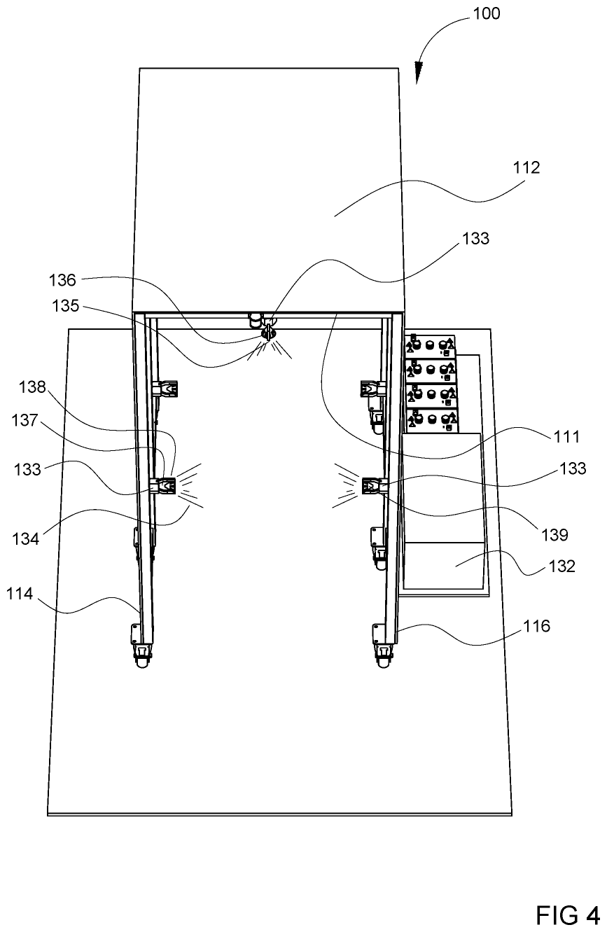

Referring now more specifically to the drawings by numerals of reference, there is shown in FIGS. 1-4, various views of a system for disinfecting (“system” 100).

FIG. 1 shows the system 100 during an ‘in-use’ condition, according to an embodiment of the present disclosure. Here, the system 100 may be beneficial for use to disinfect or sanitize an individual's body, clothing, accessories, etc. upon entrance to a building. As illustrated, the system 100 may include an enclosure 110, a first disinfecting assembly 120, a second disinfecting assembly 130, and a power source 140 configured to provide power to the system 100. As shown, the enclosure 110 may be sized to accommodate a human-user 5.

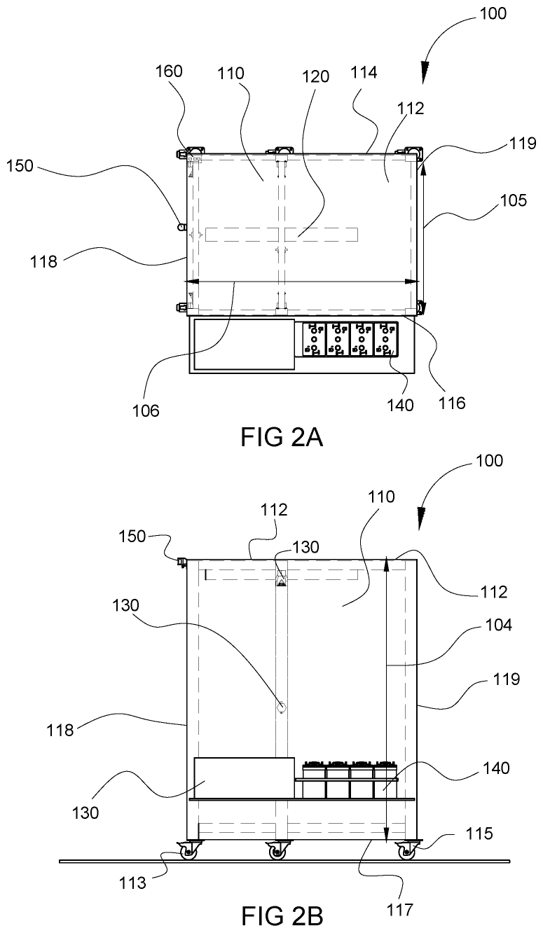

Referring now to FIGS. 2A-4 showing various views of the system 100 of FIG. 1, according to an embodiment of the present disclosure. The enclosure 110 may be defined by at least a roof 112, a first side wall 114, and a second side wall 116 opposite the first side wall 114. In some embodiments, the enclosure 110 may further include a floor 117. Further, as shown, the enclosure 110 may include an entrance 118 and an exit 119. In use, the human-user 5 (FIG. 1) may enter the enclosure 110 via the entrance 118 and exit 119 via the exit 119 when disinfection is completed. The entrance 118 and the exit 119 may be opposite each other. In some embodiments, the enclosure 110 may include a height 104 of between 7-8 feet, a width 105 of between 5-7 feet, and a length 106 of between 5-7 feet.

The enclosure 110 may include a frame defining the roof 112, first side wall 114 and the second side wall 116. The frame may include cross bracing and tube framing which may be made from aluminum material. Further, in some embodiments, at least the roof 112 may include a flame-resistant board material. The enclosure 110 may include at least four casters 113 attached to an outer surface of the floor 117, or a bottom of the enclosure 110, and configured to provide portability to the system 100. Each of the four casters 113 may include a locking brake 115 to provide stability to the human-user 5 when they are stood in the enclosure 110.

The system 100 may include at least one motion sensor 150 configured to detect the human-user 5. Further, the system 100 may include a controller 160 disposed within the enclosure 110 and configured to facilitate communication between the at least one motion sensor 150, the first disinfecting assembly 120, and the second disinfecting assembly 130. In some embodiments, upon detection of the human-user 5, the at least one motion sensor 150 may be configured to send a sensor-signal to the controller 160, and upon receipt of the sensor-signal from the at least one motion sensor 150, the controller 160 may be configured to actuate at least one of the first disinfecting assembly 120 and the second disinfecting assembly 130.

As shown, the at least one motion sensor 150 may be located about the entrance 118 of the enclosure 110. In this embodiment, the at least one motion sensor 150 may detect the human-user 5 as they walk through the entrance 118 of the enclosure 110, and actuate either the first disinfecting assembly 120, the second disinfecting assembly 130 (or both), to disinfect the human-user 5. As above, the power source 140 may be provided to power the system 100. The power source 140 may be at least one battery or other suitable means such as AC. The at least one battery may be disposable, or rechargeable.

The first disinfecting assembly 120 may be attached about an inner surface of the enclosure 110. Preferably, the first disinfecting assembly 120 may be attached to the inner surface 111 on the roof 112 of the enclosure 110. As illustrated, the first disinfecting assembly 120 may be located along the length 106 of the enclosure 110 to provide the human-user 5 with maximum exposure. The first disinfecting assembly 120 may include a light source 122 emitting shortwave ultraviolet light 124 configured to disinfect at least a portion of the human-user 5. The shortwave ultraviolet light 124 may disinfect via ultraviolet germicidal irradiation. The portion may be a piece of clothing, an accessory, a body part, a whole body of the human-user 5, etc.

The light source 122 may include an ultraviolet light-emitting diode (UVC LED) lamp. The shortwave ultraviolet light 124 may include a wavelength 106 between 255-280 nm. In other embodiments, the light source 122 may be a mercury-based lamp, a pulsed-xenon lamp, and the like. The light source 122 may be contained within a protective housing. Those with ordinary skill in the art will now appreciate that upon reading this specification and by their understanding the art of ultraviolet germicidal irradiation as described herein, methods of ultraviolet disinfection will be understood by those knowledgeable in such art.

The second disinfecting assembly 130 may include a receptacle 132 housing a disinfecting solution 134 and a dispensing means 135 in communication with the receptacle 132. The receptacle 132 may include a capacity configured to house 15 gallons of the disinfecting solution 134. The dispensing means 135 may be configured to selectively dispense a predetermined amount of disinfecting solution 134 over the at least said portion of the human-user 5 (FIG. 1). The second disinfecting assembly 130 may be located about the inner-surface 111 of the enclosure 110.

Preferably, the dispensing means 135 may include at least one spray nozzle 138. In some embodiments, the at least one spray nozzle 138 may include three spray nozzles 133. A first spray nozzle 136 of the three spray nozzles 133 may be attached to the inner-surface 111 on the roof 112 of the enclosure 110, a second spray nozzle 137 of the three spray nozzles 133 may be attached to the inner-surface 111 on one of the first side wall 114 and the second side wall 116 of the enclosure 110, and a third spray nozzle 139 of the three spray nozzles 133 may be attached to the inner-surface 111 on another one of the first side wall 114 and the second side wall 116 of the enclosure 110. In this embodiment, the human-user 5 (FIG. 1) may enter the enclosure 110, and the three spray nozzles 133 may be dispensed onto sides and head of the human-user 5 (FIG. 1).

The disinfecting solution 134 may be a liquid. In this embodiment, the at least one spray nozzle 138 may be configured to dispense the liquid as an atomized mist. In other embodiments, the disinfecting solution 134 may be a gas, solid, powder, and the like. It should be noted that, under appropriate circumstances, considering such issues as design preference, user preferences, marketing preferences, cost, structural requirements, available materials, technological advances, etc., other methods for disinfection are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

1. A system for disinfecting comprising:
an enclosure defined by at least a roof, a first side wall, and a second side wall opposite the first side wall, the enclosure further including an entrance and an exit, the enclosure being sized to accommodate a human-user;
a first disinfecting assembly attached about an inner-surface of the enclosure, the first disinfecting assembly including a light source emitting shortwave ultraviolet light, the shortwave ultraviolet light configured to disinfect at least a portion of the human-user;
a second disinfecting assembly located about the inner-surface of the enclosure, the second disinfecting assembly including a receptacle housing a disinfecting solution, and a dispensing means in communication with the receptacle, the dispensing means configured to selectively dispense a predetermined amount of disinfecting solution over the at least said portion of the human-user; and
a power source configured to provide power to the system.

2. The system of claim 1, further comprising at least one motion sensor configured to detect the human-user.

3. The system of claim 2, wherein the at least one motion sensor is located about the entrance of the enclosure.

4. The system of claim 3, further comprising a controller disposed within the enclosure, the controller being configured to facilitate communication between the at least one motion sensor, the first disinfecting assembly, and the second disinfecting assembly.

5. The system of claim 4, wherein upon detection of the human-user, the at least one motion sensor is configured to send a sensor-signal to the controller, and wherein upon receipt of the sensor-signal from the at least one motion sensor, the controller is configured to actuate at least one of the first disinfecting assembly and the second disinfecting assembly.

6. The system of claim 1, wherein the first disinfecting assembly is attached to the inner surface on the roof of the enclosure.

7. The system of claim 1, wherein the dispensing means of the second disinfecting assembly includes at least one spray nozzle.

8. The system of claim 7, wherein the at least one spray nozzle includes three spray nozzles.

9. The system of claim 8, wherein a first spray nozzle of the three spray nozzles is attached to the inner surface on the roof of the enclosure, wherein a second spray nozzle of the three spray nozzles is attached to the inner surface on one of the first side and the second side of the enclosure, and wherein a third spray nozzle of the three spray nozzles is attached to the inner surface on another one of the first side wall and the second side wall of the enclosure.

10. The system of claim 9, wherein the disinfecting solution is a liquid.

11. The system of claim 10, wherein the at least one spray nozzle is configured to dispense the liquid as an atomized mist.

12. The system of claim 1, wherein the enclosure further includes a floor.

13. The system of claim 12, wherein the enclosure further includes at least four casters attached to an outer surface of the floor and configured to provide portability to the system.

14. The system of claim 13, wherein each of the four casters includes a locking brake.

15. The system of claim 1, wherein at least the roof of the enclosure comprises a flame-resistant board material.

16. The system of claim 1, wherein the enclosure includes a height of between 7-8 feet.

17. The system of claim 16, wherein the enclosure includes a width of between 5-7 feet.

18. The system of claim 17, wherein the enclosure includes a length of between 5-7 feet.

19. A system for disinfecting comprising:
an enclosure, the enclosure being defined by at least a roof, a floor opposite the roof, a first side wall, and a second side wall opposite the first side wall, the enclosure further including an entrance and an exit, the enclosure being sized to accommodate a human-user;
at least four casters attached to an outer surface of the floor, each of the four casters including a locking brake, the at least one four casters being configured to provide portability to the system;
a first disinfecting assembly attached to an inner-surface of the enclosure, the first disinfecting assembly including a light source emitting shortwave ultraviolet light, the shortwave ultraviolet light configured to disinfect at least a portion of the human-user;
a second disinfecting assembly located about the inner-surface of the enclosure, the second disinfecting assembly including a receptacle housing a disinfecting solution, and a dispensing means in communication with the receptacle, the dispensing means configured to selectively dispense a predetermined amount of the disinfecting solution over the at least said portion of the human-user;
at least one motion sensor located about the entrance of the enclosure, the at least one motion sensor configured to detect the human-user;
a controller disposed within the enclosure, the controller being configured to facilitate communication between the at least one motion sensor, the first disinfecting assembly, and the second disinfecting assembly, wherein upon detection of the human-user, the at least one motion sensor is configured to send a sensor-signal to the controller, and wherein upon receipt of the sensor-signal from the at least one motion sensor, the controller is configured to actuate at least one of the first disinfecting assembly and the second disinfecting assembly; and
a power source configured to provide power to the system.

20. A system for disinfecting comprising:
an enclosure, the enclosure being defined by at least a roof, a floor opposite the roof, a first side wall, and a second side wall opposite the first side wall, the enclosure further including an entrance and an exit, the enclosure being sized to accommodate a human-user;
at least four casters attached to an outer surface of the floor, each of the four casters including a locking brake, the at least one four casters being configured to provide portability to the system;
a first disinfecting assembly attached to an inner-surface on the roof of the enclosure, the first disinfecting assembly including a light source emitting shortwave ultraviolet light, the shortwave ultraviolet light configured to disinfect at least a portion of the human-user;
a second disinfecting assembly including a receptacle housing a liquid disinfecting solution, and three spray nozzles in communication with the receptacle, a first spray nozzle of the three spray nozzles attached to the inner surface on the roof of the enclosure, a second spray nozzle of the three spray nozzles attached to the inner surface on one of the first side and the second side of the enclosure, a third spray nozzle of the three spray nozzles attached to the inner surface on another one of the first side wall and the second side wall of the enclosure, the three spray nozzles collectively configured to selectively dispense a predetermined amount of the liquid disinfecting solution as an atomized mist over the at least said portion of the human-user;
at least one motion sensor located about the entrance of the enclosure, the at least one motion sensor configured to detect the human-user;
a controller disposed within the enclosure, the controller being configured to facilitate communication between the at least one motion sensor, the first disinfecting assembly, and the second disinfecting assembly, wherein upon detection of the human-user, the at least one motion sensor is configured to send a sensor-signal to the controller, and wherein upon receipt of the sensor-signal from the at least one motion sensor, the controller is configured to actuate at least one of the first disinfecting assembly and the second disinfecting assembly; and
a power source configured to provide power to the system.
